(19) United States
(12) Patent Application Publication
(10) Pub. No.: US 2013/0343511 A1
(43) Pub. Date: Dec. 26, 2013

(54) Quantitative Two-Dimensional Fluoroscopy via Computed Tomography

(71) Applicants: Himanshu P. Shukla, Lafayette, CA (US); Supratik Bose, San Ramon, CA (US)

(72) Inventors: Himanshu P. Shukla, Lafayette, CA (US); Supratik Bose, San Ramon, CA (US)

(73) Assignee: Siemens Medical Solutions USA, Inc., Malvem, PA (US)

(21) Appl. No.: 13/532,324

(22) Filed: Jun. 25, 2012

(51) Int. Cl. A61B 6/03

(52) U.S. Cl. 378/6

(57) ABSTRACT

A system includes obtaining of a reference projection image of a target volume at an isocenter of a computed tomography scanner; obtaining of a plurality of two-dimensional fluoroscopic images by the computed tomography scanner of at least a portion of the target volume at the isocenter of the computed tomography scanner; displaying the reference projection image and the plurality of two-dimensional fluoroscopic images in a combined view; measuring a two-dimensional contour of a projection of a movement of the target volume in the combined view; and determining a true contour of the movement in a plane containing a point-of-interest within the target volume based on the two-dimensional contour of the projection of the movement.

### BACKGROUND

1. Field

The embodiments described below relate generally to imaging using X-rays. More specifically, some embodiments relate to quantitative two-dimensional imaging using a computed tomography scanner.

2. Description

Imaging using radiation imaging beams is used for diagnostic purposes and in planning and administering radiotherapy treatment plans, including for example pre-planning processes of a radiotherapy treatment session.

One conventional method for imaging is wide area detector based fluoroscopy using a conventional X-ray system. Such a system may employ an X-ray source and a wide area radiation detector that is typically large enough to fully capture a patient area being imaged. This type of system may cover a large area of the patient such that a moving target area and reference anatomy of the patient may both be imaged from a fixed position without moving the patient or the couch supporting the patient. Another conventional imaging system includes four-dimensional computed tomography (4D CT) for the acquisition of images. Such a system may provide a high quality and accurate temporal 3D volume of an imaged target volume. A 4D CT acquisition system typically requires a regular periodic motion of the target volume. However, regular periodic motion of a target volume may be rare in practice. Additionally, most such systems typically require significant computation resources, may need an external proxy signal, and the significant imaging dose and data burden needed to obtain the detailed temporal 3D data may not justify the desired imaging gains provided by the 4D CT imaging system.

Improved computed tomography (CT) scanner based imaging of a moving target volume is desired, with the imaging being sufficiently accurate to provide efficient quantitative measures of a moving target volume imaged thereby.

### SUMMARY

In order to address the foregoing, some embodiments provide a system, method, apparatus, and means for obtaining a reference projection image of a target volume with a computed tomography scanner; acquiring a plurality of two-dimensional fluoroscopic images by the computed tomography of at least a portion of the target volume with the computed tomography scanner; displaying the reference projection image and the plurality of two-dimensional fluoroscopic images in a combined view, the reference projection image forming a background of the combined view and the plurality of two-dimensional fluoroscopic images forming a foreground of the combined view; ascribing and measuring a two-dimensional contour of a projection of a movement of the target volume in the combined view; and determining a true contour of the movement at a point-of-interest POI off the target volume based on the two-dimensional contour of the projection of the movement.

The appended claims are not limited to the disclosed embodiments, however, as those in the art can readily adapt the descriptions herein to create other embodiments and applications.

### BRIEF DESCRIPTION OF THE DRAWINGS

Embodiments will become readily apparent from consideration of the following specification as illustrated in the accompanying drawings, in which like reference numerals designate like parts, and wherein:

FIG. 1 is a perspective view of a treatment room according to some embodiments;

FIG. 2 is a block diagram illustrating elements of a computed tomography system, in accordance with some embodiments;

FIGS. 3A and 3B are flow diagrams of processes, in accordance with some embodiments herein;

FIG. 4 is an illustrative depiction of a system for acquiring two-dimensional fluoroscopic images with a computer tomography scanner, in accordance with some embodiments;

FIG. 5 is an illustrative depiction of a system for acquiring two-dimensional fluoroscopic images with a computer tomography scanner, including a target volume according to some embodiments;

FIG. 6 is an illustrative depiction of an apparatus for displaying and evaluating of two-dimensional fluoroscopy acquired with a computer tomography scanner according to some embodiments herein; and

FIGS. 7A-7D illustrate a method of updating a contour margin according to some embodiments.

### DETAILED DESCRIPTION

The following description is provided to enable a person in the art to make and use some embodiments and sets forth the best mode contemplated by the inventors for carrying out some embodiments. Various modifications, however, will remain readily apparent to those in the art.

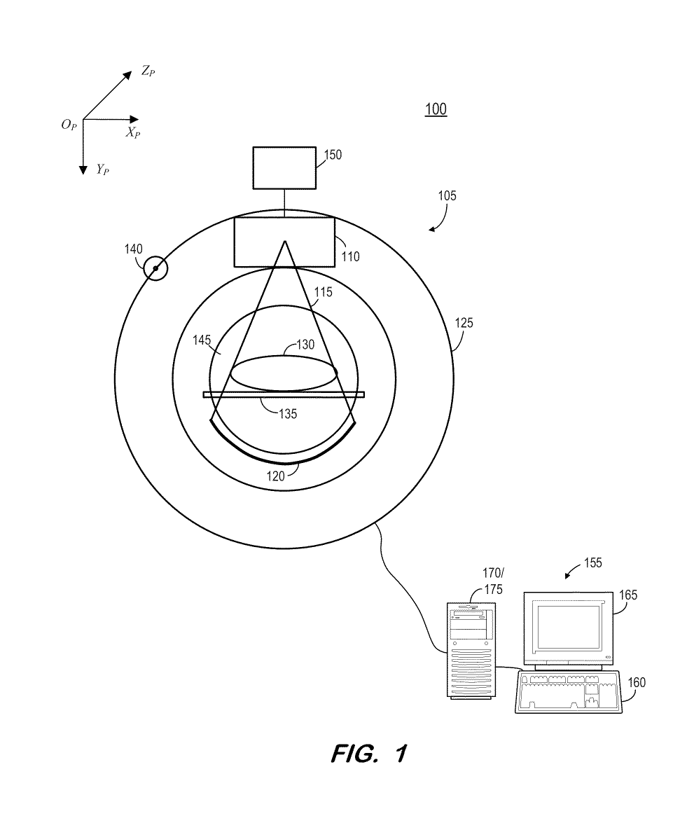

FIG. 1 illustrates computed tomography (CT) scanner 105 located in a CT room 100. CT scanner 105 includes X-ray source 110 for emitting, for example, a fan-shaped X-ray beam 115 toward radiation detector 120. Both X-ray source 110 and radiation detector 120 are mounted on ring 125 such that they may be rotated through 360 degrees while maintaining the physical relationship therebetween (e.g., source-to-detector distance) throughout stationary and rotational movements of the source and detector. Each of the devices shown in FIG. 1 may include fewer or more elements than those shown and are not limited to the devices shown in FIG. 1.

In operation, patient 130 may be positioned on couch 135 to place a portion of patient 130 between X-ray source 110 and radiation detector 120. In some aspects, X-ray source 110 and detector 120 may be rotated by rotation drive 140 around cavity 145 in which patient 130 is position and supported by couch 135. X-ray source 110 is powered by high-voltage generator 150 to transmit imaging X-ray radiation toward detector 120. Detector 120 receives the radiation and produces a set of data (i.e., a projection image) for a given projection angle of the X-ray source.

Operator system or computer system 155 includes an input device 160 for receiving instructions from a user operator. Input device 160 may include one or more of any type of user input device including, but not limited to, a keyboard, a keypad, a joy stick, a touchpad, a touchscreen, a voice-input system, and other user input elements. Output device 165 may include a monitor for presenting operational parameters of CT scanner 105. Output device 165 may also display images acquired, processed, and/or used by CT scanner 105. Input device 160 and output device 165 are coupled to processor 170 and storage 175. Processor 170 may execute program code stored in storage 175 to perform any of the operations, processes, methods, and/or to cause CT scanner 105 to perform any of the operations, processes, and methods described herein.

Processor 170 may execute program code of storage 175 to reconstruct three-dimensional images from the projection images acquired by scanner 105 (i.e., 3DCT). Processor 170 may also allow an operator to view, on display 165, “slices” of such three-dimensional image. Processor 170 may also execute program code of storage 175 to allow an operator to identify a treatment or target isocenter within patient 130.

During radiation emissions X-ray source 110 may emit a beam of kilovoltage x-rays along a beam axis towards detector 120. The beam is emitted towards the isocenter of CT scanner 105. Due to a divergence of the emitted beam and/or the shaping of the beam by beam-shaping devices in some embodiments, the beam will deliver radiation to a volume of patient 130 rather than only through the CT isocenter. The various components of radiotherapy treatment room 100 may be used to image an object such as patient 130. The patient may be positioned on couch 135 for imaging by CT scanner 105. CT scanner 105 may be used to acquire a set of two-dimensional fluoroscopy images of a target volume within patient 130 positioned between X-ray source 110 and detector 120. The fluoroscopy images may include multiple images acquired over multiple cycles of a breathing or other motion. The elements of treatment room 100 may be employed in other applications according to some embodiments.

Couch 135 may support a patient during radiation imaging and other aspects discussed herein. Couch 135 may be adjustable to assist in positioning patient 130 or a particular target area and volume of the patient between X-ray source 110 and detector 120. Table 135 may be limited to moving in the Z-direction, in and out of CT cavity 145 according to the reference coordinate system of FIG. 1 (i.e., into and out of the plane of the page). In some embodiments, table 135 may be selectively moved during at least portions of a radiation imaging session, in accordance with processes herein.

Some embodiments herein include monitoring a motion of a targeted patient area, for radiotherapy treatment, diagnostic, and other purposes, using real-time imaging acquired by CT scanner 105. In some aspects, three-dimensional CT (3D CT) acquisition that provides a high quality accurate 3D CT volume of a patient target area, from which digitally reconstructed radiographs (DRR) may be generated. As used herein, a DRR is a fully divergent projection.

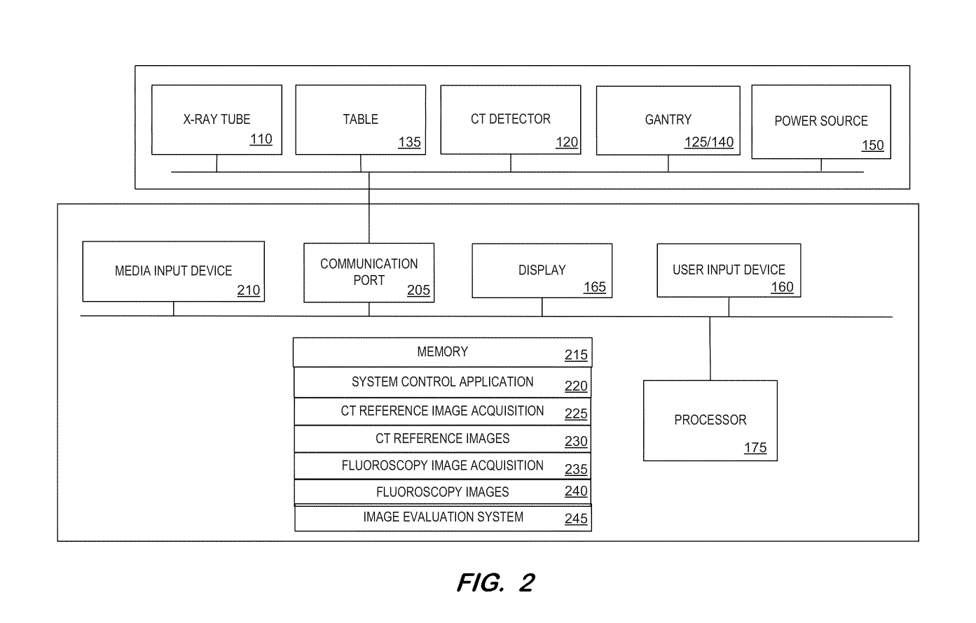

FIG. 2 is a block diagram of CT system 100 according to some embodiments, including CT scanner system 105 and computer system 155 discussed above in connection with FIG. 1. Components introduced in the description of FIG. 1 are numbered similarly in FIG. 2. The illustrated elements may be implemented by any suitable combination of hardware, software and/or firmware. Computer system 155 may include one or more separate computing devices or systems.

Computer system 155 includes communication port 205 for interfacing with CT scanner 105. Computer system 155 may issue commands and requests for controlling various elements of CT scanner 105 and may receive feedback (i.e., responses) therefrom via communication port 205. Computer system 155, may issue a command via communication port 205 to control X-ray tube 110 to emit a radiation beam towards CT detector 120 to acquire projections, and may receive the projections from CT scanner 105 over communication port 105.

Communication port 205 may comprise any type of interface suitable for receiving data from CT scanner 105. Communication port 205 may comprise a proprietary interface associated with, for example, a manufacturer of CT scanner 105. Computer system 155 is shown as including media input device 235 that may comprise a mechanism for receiving a non-transitory storage medium and reading/writing data from/to the medium.

Display 165 may comprise any one or more devices for displaying images and control interfaces to a user/operator of computer system 155. Display 165 may display images such as any projections and two-dimensional images acquired or generated by CT scanner 105 and/or any fluoroscopic images generated by CT scanner 105 according to some embodiments herein. User input device 160 may be operated by the user to input data and commands to computer system 155. User input device 160 may comprise any input device or devices that are or become known.

Processor 175 executes processor-executable code stored in memory 255 to accomplish operations and steps of processes disclosed herein by CT system 100, according to some embodiments. In some aspects, memory 255 may store program code that may be executed by processor 175. The program code may embody instructions for implementing processes herein. The stored code may include system control application 220 (i.e., an operating system), CT image acquisition program 225 to acquire CT projections and CT fluoroscopy image acquisition program 235 to acquire 2D fluoroscopic images (e.g., a dataset of two-dimensional images over a period of time) according to some embodiments. Memory 255 may also store CT images 230 and fluoroscopic images 235 generated and/or used by CT scanner 100.

In some aspects, object 130 (e.g., a patient's body) may be positioned on couch 135 to place at least a portion of the patient between X-ray source 110 and radiation detector 120. Next, X-ray tube 110 and detector 120 are rotated by rotation drive 140 around cavity 125 in which the target object 130 lies to a desired angle. The X-ray tube 110 is powered by high-voltage generator 150 to transmit X-ray radiation toward detector 120 with X-ray tube 110 and radiation detector 120 at a static, fixed position. Detector 120 receives the radiation and produces a projection image for each projection angle.

Each projection image includes a set of data that represents the attenuative properties of tissues along divergent lines between X-ray tube 110 and detector 120. The projection images are transmitted to computer system 155. For generation of a three-dimensional image, computer system 155 calculates attenuation coefficients (e.g., Hounsfield numbers) of predetermined points based on the projection images. The attenuation coefficients are used to generate a three-dimensional image representing the portion of patient 130 that is positioned between X-ray source 110 and CT detector 120.

The dimensions of the projection images are similar to the dimensions of detector 120 on which detecting elements reside. Referring to the example of FIG. 1, the dimension on which the detecting elements reside in the illustrated X-direction (i.e., width of couch 135) may be sufficient to capture most of the radiation that passes through patient 130 in the X-direction. Detector 120 may comprise multiple (e.g., six through six hundred) rows of detecting elements in the illustrated Z-direction (i.e., into and out of the plane of FIG. 1). Accordingly, the acquired projection images may, for example, extend few or many centimeters in the Z-direction for any one position of couch 135.

In some embodiments, couch 120 may be moved in the Z-direction to place a different portion of patient 130 between X-ray tube 110 and radiation detector 120 in the instance a full extent of the desired target area of the patient is not captured by a single positioning of the patient due to the dimensions of the CT detector. A two-dimensional image of the different portion(s) may be acquired as described above.

A hardware environment according to some embodiments may include fewer, more, or alternative elements than those shown in FIGS. 1 and 2. Embodiments of the present disclosure are not limited to the devices and/or to the specifically illustrated environment of the figures. For example, some embodiments may include another type of image acquisition device to acquire projections.

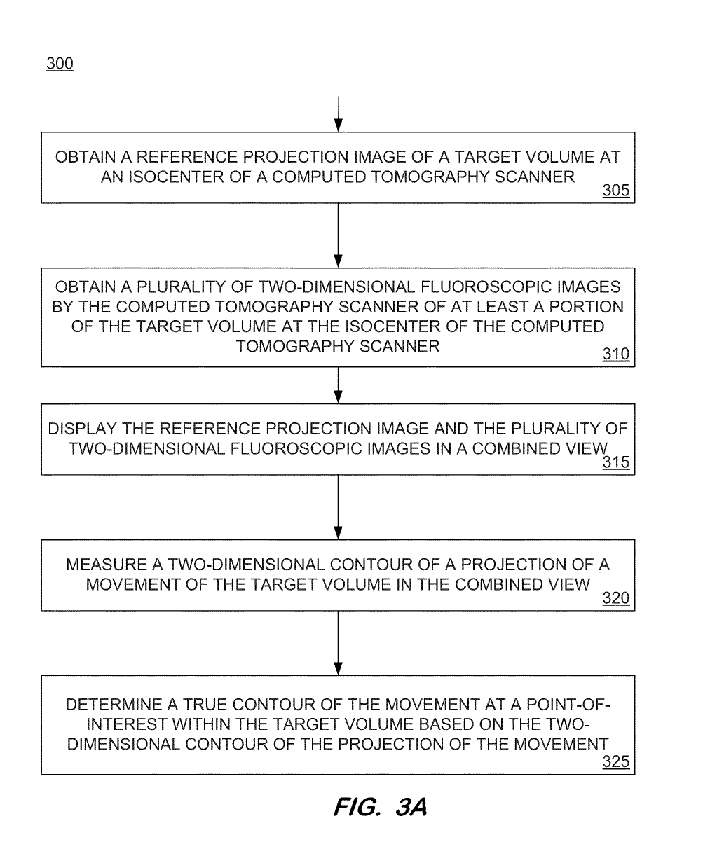

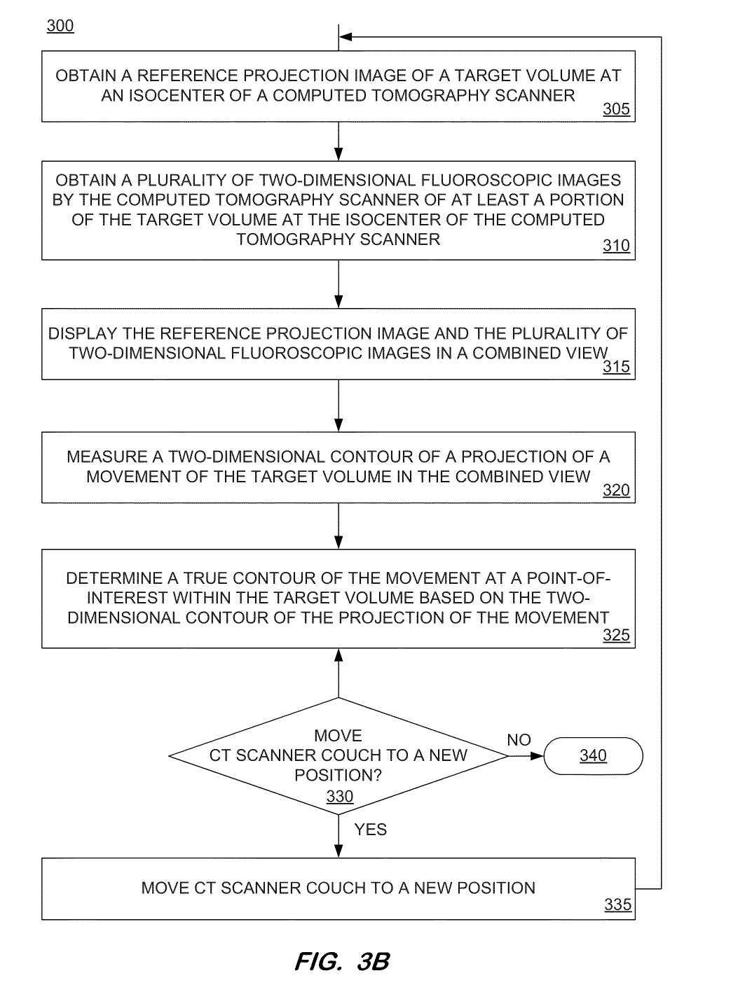

FIG. 3 is a flow diagram of process 300 according to some embodiments. Process 300 and the other processes described herein may be performed using any suitable combination of hardware, software or manual means. Software embodying these processes may be stored by any tangible, non-transitory medium, including but not limited to a hard disk drive, a solid-state drive, a CD-ROM, a DVD-ROM, a flash drive, and other types of storage devices. Examples of these processes will be described hereinbelow with respect to the elements of systems 100 and 200, yet embodiments are not limited thereto.

At operation S305, a reference projection image of a target volume is obtaine. In some embodiments, the reference projection image is obtained by CT scanner 105 with couch 135 at a fixed position. In this manner, the geometric configuration between the X-ray source 110, detector 120, and the target volume on couch 135 are static during the obtaining of the reference projection image. In some embodiments, the reference projection image may be obtained using a CT scanner such as scanner 105 of FIG. 1. Some embodiments of S305 may not include acquisition of projection images and reconstruction of a two-dimensional image based on the projection images by CR scanner 105, but may instead include obtaining a two-dimensional image that was created by another entity.

In some embodiments, the reference projection image may comprise a CT projection radiograph (e.g., a “topogram”, “scanogram”, “scout”, “pilot”, etc. . . . ) or a digitally reconstructed radiograph (i.e., a DRR). The reference projection image of S305 may be generated by image(s) obtained during process 300 or by image(s) generated prior to process 300. In some aspects, the reference projection image may be constructed based on a 3DCT volume acquired by CT scanner 105 (or another entity). The 3DCT volume may be obtained prior to an initiation of process 300 such that the 3DCT data is available for use by process 300. The 3DCT volume provides an accurate and precise reconstruction of selected patient volume (e.g., patient 130).

It is noted that the reference projection image of the target volume is a projection of the target volume.

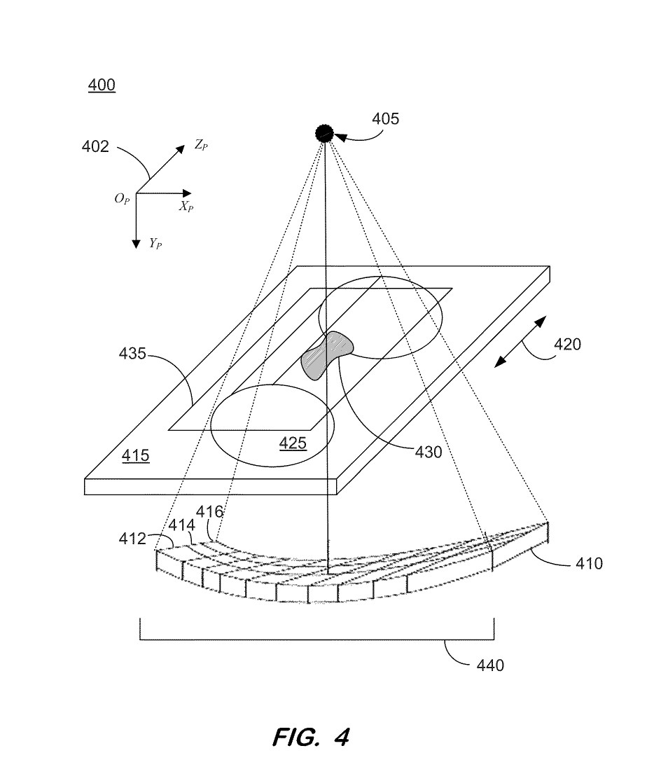

Referring to FIG. 4, an illustrative depiction of a system 400 for acquiring CT images and two-dimensional fluoroscopic images with a CT scanner is shown, in accordance with some embodiments herein. As illustrated in FIG. 4, CT scanner system 400 includes an X-ray source 405 and a detector 410 arranged in a fixed distance relationship to each other. Positioned between the X-ray source 405 and detector 410 is couch 415 for supporting a target volume 425 (e.g., a patient). Couch 415 may be selectively moved in the Z-direction as indicated by arrow 420, while remaining fixed in the X-direction and Y-direction with respect to the patient coordinate system 402 attached to the patient volume.

In some embodiments, a reference projection image is constructed by projecting an image of a target volume from detector 410 onto a flat plane 435 that passes through the CT scanner isocenter. Acquisition of the reference projection image may be accomplished by, for example, a CT radiograph (e.g., a “topogram”) and a DRR (and other mechanisms), wherein the dimensions of projection of objects is dependent on the objects' relative position in the imaging geometry between the source (e.g., 405) and the detector (e.g., 410). In some embodiments, the projection image(s) may be acquired with the couch of the CT scanner at a fixed position or very quickly such that a movement of the couch approaches a fixed position during the imaging operation.

At operation S310, a plurality of two-dimensional (2D) fluoroscopic images of at least a portion of the target volume are acquired by the CT scanner. Detector 410 comprises a plurality of columns 412, 414, and 416 for detecting radiation emitted from X-ray source 405. Columns 412, 414, and 416 are parallel to a width 400 of couch 410. The width of the detector columns may be relatively narrow (e.g., 2 cm-8 cm) and projection images may be constructed by projecting pixel data from a single column. In some aspects, the 2D fluoroscopic images acquired by CT (“2Dfluoro_{CT}”) are acquired with the couch at a fixed position or moved in effectively discrete steps at which the images are obtained. At each step or position of the couch, multiple frames covering multiple motion cycles of the target volume may be obtained. In this manner, projection images including a comprehensive representation of the motion of the target volume may be acquired.

In some aspects, a full extent of the motion of the target volume may not be completely projected in a dataset of fluoroscopic images for the couch at any one position. Factors related to whether the moving target volume is completely projected within the narrow fluoroscopic strip corresponding to the narrow column of the CT detector 410 may include the size of the target volume, the magnitude of the motion of the target volume, and the dimensions of the CT detector. In some embodiments, couch 410 may be moved at S310, as needed, to capture the full extent and range of the motion of the target not captured with the couch at one position.

In some embodiments, fluoroscopic images acquired in accordance with some embodiments herein may have real-time filters and/or permanent filters applied during the fluoroscopic image acquisition process (e.g., operation S310).

At operation S315, a display of the 2Dfluoro_{CT} images of S310 and the reference projection image of S305 are displayed in combination in a common display. This common display is referred to herein as a “combined view”. The combined view herein includes, for a given fixed couch position, the corresponding “live” fluoroscopic images of at least a portion of the moving target volume acquired at that couch position and the static reference projection image of the target volume at the same couch position. In some embodiments, the fluoroscopic images are displayed in a central location of the display as the foreground in the combined view, while the static reference image is presented as the background of the combined view. In the combined view, the couch position in the reference image and the couch position in the fluoroscopic images are “matched” to each other. Accordingly, the imaging geometry of the reference image and the fluoroscopic images correlate to each other, with the projections for the reference image and the fluoroscopic images all being in the plane 435 at the isocenter of the CT scanner.

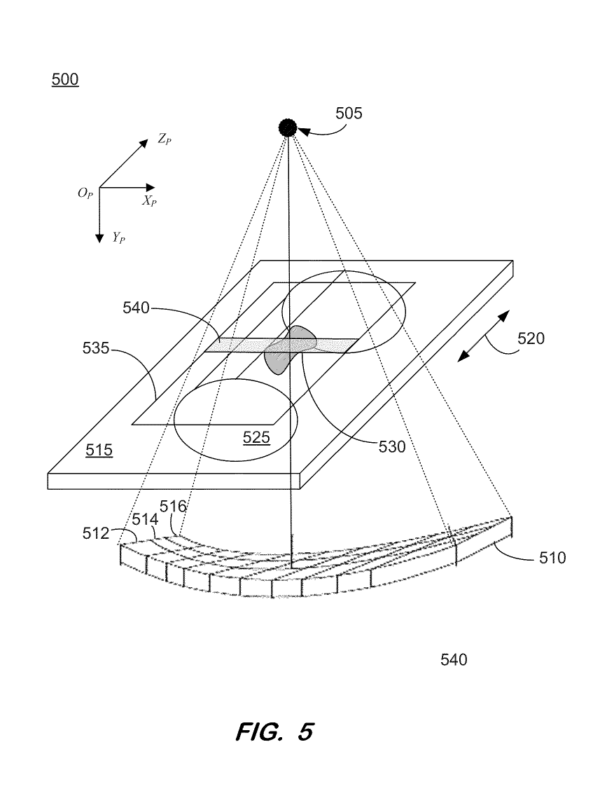

FIG. 5 is an illustrative depiction of some aspects herein, including a CT scanner use case for the example process 300 of FIG. 3. FIG. 5 is similar in some regards to FIG. 4. In particular, environment 500 includes a CT scanner X-ray source 505 and a CT detector 510 separated by a fixed distance, where the CT detector comprises a number of narrow detecting elements 512, 514, and 516. FIG. 5 further includes a patient 525 supported by couch 515 that is moveable in the Z-direction as indicated at 520. Patient 525 has a moving target volume 530 located therein for imaging, in accordance with process(es) herein. As shown, a fluoroscopic strip 540 corresponding to the narrow CT scanner detector columns is provided in the plane 535 that passes through the isocenter of the CT scanner. The plane including the isocenter of the CT scanner is the same plane including projections of the reference image and the fluoroscopic images.

It is noted that the location of the target volume may be determined from known 3DCT data regarding the target volume. As discussed above, the 3DCT data may be obtained prior to and independent of an initiation or execution of process 300. Furthermore, a user identified reference point (P01) within the target volume proximal to the moving target may be identified based on the 3DCT data. The user identified isocenter may thus be known with a high degree of accuracy and precision. In some aspects, the POI within the target volume is different from the isocenter of the CT scanner.

Returning to FIG. 3, process 300 continues to S320 where a quantitative evaluation or measure of the motion of the target volume is performed. A quantitative evaluation of the motion of the target volume may be obtained by measuring the extent of movement of the target volume in the combined view of S315, which presents “live” moving of the fluoroscopic images displayed over the static background reference image. The fluoroscopic images displayed in the foreground and the background reference image are geometrically consistent with each other since they correspond to the same source-to-detector distance (i.e., SDD), the same source-to-subject (i.e., SSD), and the same couch position. Furthermore, since the isocenter of the target volume is known a priori from the user-identified point-of-interest, POI, based on the 3DCT data, this known reference point in the 3D volume may be used to quantitatively determine, calculate, or evaluate motion in the combined view. That is, with knowledge of the depth within the subject object at which the motion of interest is occurring (e.g., the POI within the target), the motion reflected in the fluoroscopy can be determined in a quantitative and objective manner. In some regards, an exact size of a subject target volume and any motion associated therewith may be determined in an absolute, deterministic manner.

Operation S320 may include measuring a 2D contour of a projection of a movement of the target volume of the combined view from S315. In some embodiments, the measuring may be facilitated by one or more contouring tools, including for example rulers and graphic overlays to accurately measure aspects of the 2Dflouoro_{CT} in the combined view. Some aspects of the measuring and evaluating of S320 may be automatically invoked and/or performed, whereas some aspects may be selectively engaged and performed in response to user input(s). In some embodiments, a contouring tool may include functionality to ascribe, draw, or trace a line or other indicator on a displayed presentation of the combined view. The location of the ascribed or drawn line and/or indicator may be used to generate measurements consistent with the accuracy of the reference image(s).

For example, a combined view comprising a reference image based on a “topogram” may be used to obtain qualitative measurements or evaluations of the movement of the target volume. The accuracy and detail of information in the “topogram” (i.e., two-dimensional) used in the combined view of this example may be sufficient for making qualitative evaluations of whether a single CT scanner couch position is adequate to cover the extent of motion of relevant target volume, whether the movement is regular (e.g., periodic), etc.

A combined view comprising a reference image based on a DRR may be used to obtain quantitative measurements or evaluations of the movement of the target volume. The accuracy and detail of information in the DRR (i.e., a POI within the target volume) used in the combined view of this example may be sufficient for making quantitative evaluations, as will be discussed in detail below.

In some embodiments, a tool utilized in S320 for measuring and evaluation purposes may be activated, either automatically or otherwise, when the CT scanner couch is at a fixed location. In this aspect, accuracy of the evaluation may be accomplished in a deterministic manner without undue complexity of calculations.

In accord with aspects of process 300, a true contour of the movement of the target volume at the POI of the target volume may be determined at operation S325. The true contour at the target POI 325 may be determined based on the 2D contour of the projection of the target's movement. Due to the known location of the user-identified POI and its location within the reference projection image based on, for example 3DCT data associated with the target volume, a contour drawn on the plane of the combined view (i.e., the plane including the reference image (e.g., a DRR) and the fluoroscopic images at the isocenter of the CT scanner) may be used to determine the corresponding contour at the plane including the POI that is parallel to the plane of the combined view. In some aspects, operation S325 may back-project the contour drawn on the plane at the CT scanner isocenter including the combined view to a plane parallel thereto and at the POI of the target volume. The resultant shape or contour at the POI will accurately capture the real or “true” extent of the subject target volume in the plane including the isocenter of the POI. This resultant contour is referred to herein as the “true” contour.

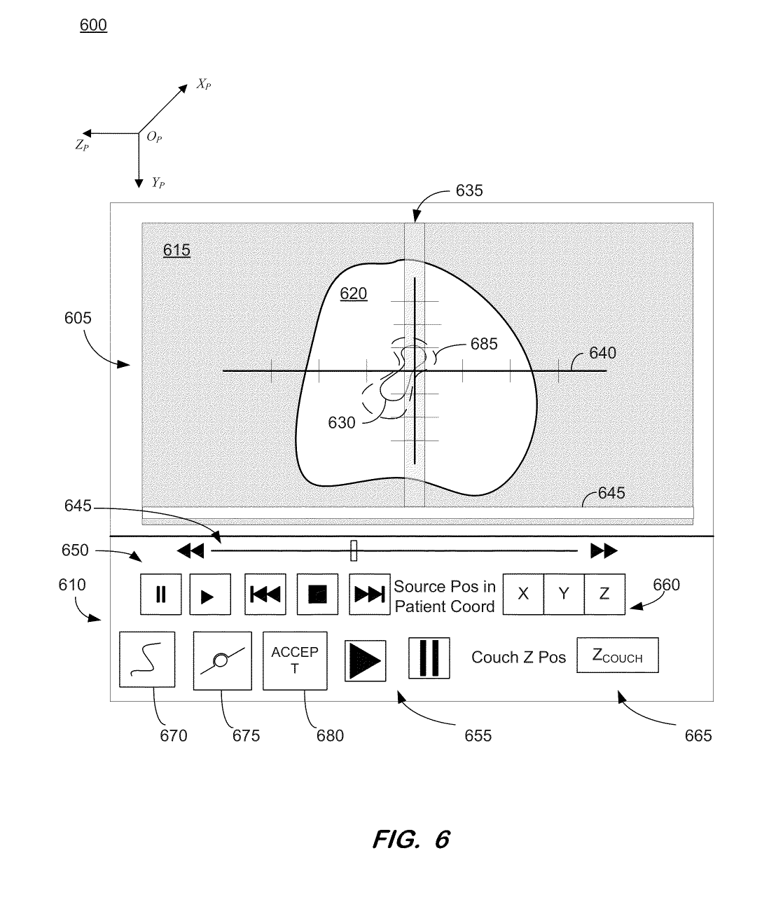

FIG. 6 is an illustrative depiction of device 600 including a combined view 605 in display area and user interface controls at 610. The combined view 605 includes a background reference image 615 that includes portions of a patient 620 and a target volume 630 within the patient, while in a foreground of the combined view fluoroscopic images acquired in accord with embodiments herein are displayed in a central strip 635. The central strip 635 may be presented with a contrasting color, intensity, and/or clarity than the background reference image 615. In this regard, the motion depicted in the fluoroscopic images in combined view 605 may be highlighted for ease of viewing and evaluation.

Combined image 605 further includes a number of reference rulers 640 for measuring and evaluating a motion exhibited by the target volume captured by the fluoroscopic images at 635. The rulers may be used alone or in conjunction with one or more contouring tools such as those controllable via user interface controls at 610 to, for example, draw a line or otherwise mark with an indicator an extent of motion depicted in the combined view.

User interface controls at 610 may include more, fewer, and alternative controls than those explicitly shown in FIG. 6. Referring to the example of FIG. 6, there is a timeline 645 that provides an indication of the progress of a display of a set of fluoroscopic images. In some aspects, the marker on the timeline 645 may be manipulated to alter a playback of the dataset of fluoroscopic images. In some regards, controls 650 may be used to control the playback of fluoroscopic images via actuators to play, fast forward, reverse, pause, and stop the playback.

In some embodiments, controls 655 may be used to selectively control a positioning of the couch of the CT scanner. These controls may be used to advance the couch from, for example, a first position to a next one or more positions. The one or more positions may be selected or determined in an effort to acquire and display images, both reference and fluoroscopic, that capture a full extent of the motion associated with the target volume. The coordinate position of the source for the displayed combined view may be presented at 660 and the position of the couch may be provided at 665.

User interface controls 610 may also include contour tools 670 and 675 for drawing a contour 685 on the 2D images of combined view 605. The contour 685 drawn on the combined view may be automatically transformed to generate a projection of the drawn contour, the “true” contour at the POI within and at the true location of the target volume, by virtue of a known POI identified at the target volume isocenter. In some embodiments, the drawing of the contour on the combined view may be selectively done by a user, while in some embodiments the drawing of the user contour may be, at least in part, automated and controlled by a processor. Control 680 may be used to accept a contour drawn on the combined view for evaluating (e.g., a quantitative evaluation and a quantitative evaluation), calculating, and determining of the “true” contour of the subject volume based on the contour 685 drawn on the combined view. In some aspects, an accepted contour and other inputs provided via user interface controls at 610 may be saved to a memory location for purposes of, for example, analysis, further processing, and reporting.

As discussed above and as illustrated in FIG. 6, the full extent of a subject target volume may not be fully captured by the fluoroscopic imaging process herein with the CT scanner couch at one position, depending on the size of the target volume, the motion associated with the target volume, and the size of the CT detector. In some embodiments herein, the position of the couch may be moved from a first position where reference and fluoroscopic images are acquired to a next one or more positions where additional reference and fluoroscopic images are obtained. The plurality of images may be correlated for each couch position in discrete combined view images to ensure a consistent geometric configuration between the reference images and the fluoroscopic images for each couch position and to capture a full extent of the motion of the target volume (at least the range of motion desired). As shown in FIG. 6, the target volume is larger than the central fluoroscopic strip 635.

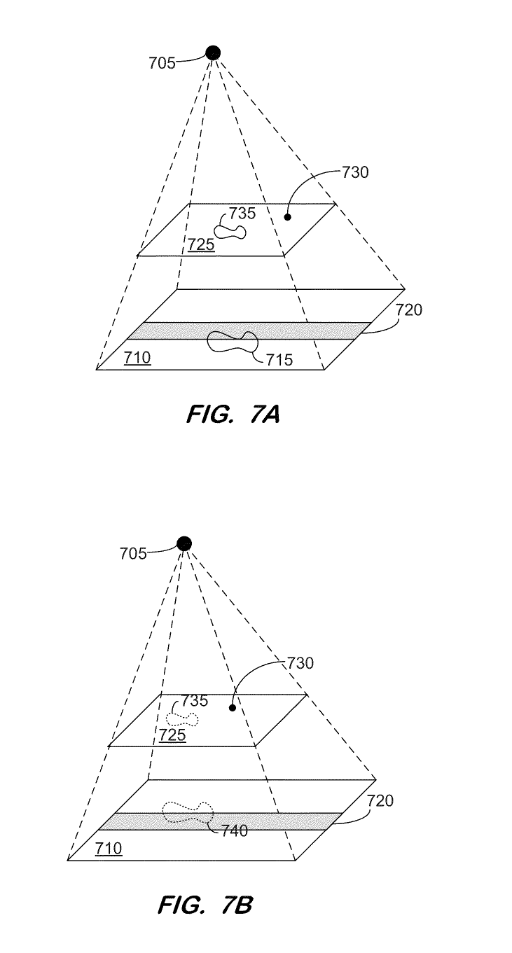

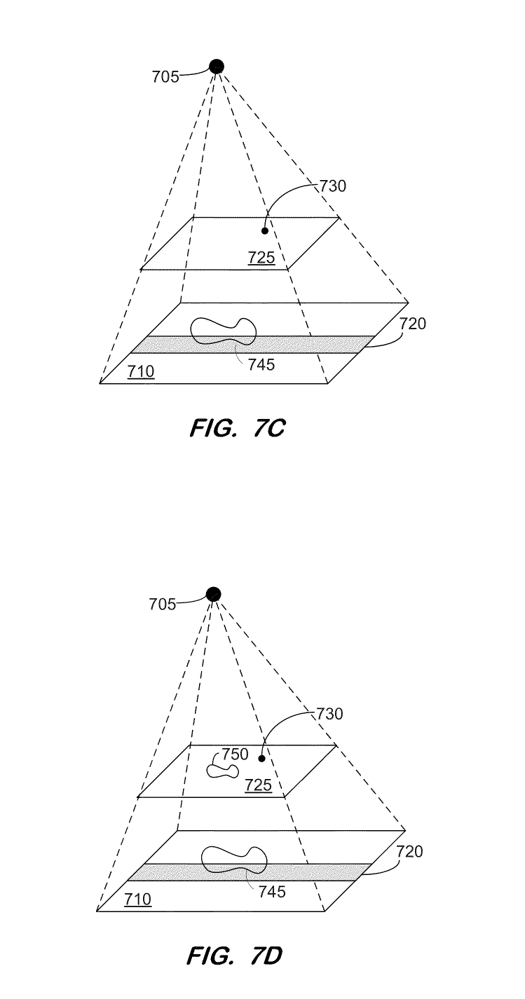

In an effort to capture the extent of the motion of the target volume, the couch of, for example FIG. 6, may be positioned at more than one location by moving the couch in the Z-direction of the CT scanner (e.g., CT scanner 100). Referring to FIGS. 7A-7D, a progression of figures illustrating aspects of an iterative method for updating a contour margin in the instance the fluoroscopic images cannot capture the full extent of the movement of the target volume at one fixed couch position is shown.

FIG. 7A discloses an environment including a CT scanner with a source at 705 for acquiring 2CDfluoro_{CT} images for a CT scanner having a couch (not shown in FIGS. 7A-7C) at a first fixed location, in accord with embodiments herein. The plane 710 passes through the isocenter of the CT scanner and includes the reference projection image (e.g., DRR, etc.) and the fluoroscopic images (cine) 720. FIG. 7A further includes a depiction of a user identified POI 730 within a target volume, based on a 3DCT volume proximal to the moving target being imaged. The plane 725 contains the real or “true” contour of the target volume and is parallel to plane 710 containing the 2D reference image and the fluoroscopic images. Given POI 730 with its known position within the imaging geometry of the reference projection image in plane 710, the contour 715 drawn on plane 710 may be back-projected to the parallel plane 725 passing through the POI 730. The resulting “true” contour 735 will thus capture the real motion of the target volume at the plane through the POI of the moving target volume.

In the event that the couch position of FIG. 7A is insufficient to capture the full extent of the motion desired to be captured within the fluoroscopy strip 720, the couch may be moved in the Z-direction to capture additional/other motion of the target volume. Such movement of the couch may be programmed into a computing device to move the couch or such movements may be decided and acted upon in an ad hoc fashion. However, as a result of moving the couch the geometric configuration of FIG. 7A between the target volume and the X-ray source changes. In particular, the position of the source changes with respect to the patient target volume and the patient coordinate system 702.

FIG. 7B shows plane 710 (i.e., CT scanner isocenter plane) and plane 725 including the POI with the couch at a second fixed position. A new reference image (e.g., a DRR) is acquired for the new couch position, as well as a new set of fluoroscopic images for the new couch position. The projections of the new reference image and the new fluoroscopy images are displayed on plane 710 through the isocenter of the CT scanner. The reference image and the images for the new couch position are correlated for consistency therebetween.

FIG. 7C illustrates a new edit of the contour projection 740 for the new (i.e., second) couch position. Given the known POI identified in the target volume, the projection of the POI in plane 710 is also known. Based on the projection of the known POI in the reference image, quantitative measures of the contour 745 may be readily determined.

The contour 745 of FIG. 7C is used to determine and generate the “true” contour 750 of the target volume as shown in FIG. 7D. The “true” contour may be generated by back-projecting the projection contour 745 to the plane including the POI 730. The “true” contour reflects the real movement of target volume in the plane 725 containing the POI.

Therefore, FIGS. 7A-7D illustrate aspects of an iterative process for determining quantitative measures or metrics for evaluating a motion of a target volume using a CT scanner with a CT couch at more than one location, in accordance with embodiments herein. The iterative process related to FIGS. 7A-7D may be implemented in some aspects by a recursive execution of operations of process 300 for each new couch position of the CT scanner, as illustrated in FIG. 3B.

FIG. 3B is an illustrative depiction of a process flow similar in some aspects to FIG. 3A. FIG. 3B includes a recursive execution of the operations of process 300 for each new couch position of the CT scanner. As indicated, the process of FIG. 3B proceeds in a manner similar to FIG. 3A until operation S330. At operation S330, a determination is made whether the CT couch is moved to a new position. The couch may be moved to a new position to place a different/additional portion of the target volume between the X-ray source and detector of the CT scanner. Moving the couch to a new position at S335 alters the CT scanner to target volume geometric configuration. Otherwise, process 300 terminates or idles at S340. Accordingly, operations S305-S325 may be repeated for the new couch position.

It is noted that for an instance where the reference image is a fully divergent projection (e.g., a DRR), operation S305 is executed per FIG. 3B. In an instance the reference image is not a fully divergent projection (e.g., a “topogram”), then operation S305 need not be executed since the same reference image used at the first couch position can be used for the same X-ray tube angle for the new (i.e., second) couch position.

Those in the art will appreciate that various adaptations and modifications of the above-described embodiments can be configured without departing from the scope and spirit of the claims. Therefore, it is to be understood that the claims may be practiced other than as specifically described herein.

What is claimed is:

1. A method comprising:
obtaining a reference projection image of a target volume at an isocenter of a computed tomography scanner;
obtaining a plurality of two-dimensional fluoroscopic images by the computed tomography scanner of at least a portion of the target volume at the isocenter of the computed tomography scanner;
displaying the reference projection image and the plurality of two-dimensional fluoroscopic images in a combined view, the reference projection image forming a background of the combined view and the plurality of two-dimensional fluoroscopic images forming a foreground of the combined view;
measuring a two-dimensional contour of a projection of a movement of the target volume in the combined view; and
determining a true contour of the movement at a point-of-interest within the target volume based on the two-dimensional contour of the projection of the movement.

2. The method according to claim 1, further comprising receiving an indication of the point-of-interest within the target volume within a three-dimensional computed tomography volume proximal to the moving target volume, the coordinates of the point-of-interest within the target volume being known.

3. The method of claim 1, wherein the reference projection image comprises at least one of a two-dimensional computed tomography projection radiograph and a fully divergent projection.

4. The method of claim 1, wherein the reference projection image and the plurality of two-dimensional fluoroscopic images are obtained at a same computed tomography scanner to target volume geometric configuration.

5. The method of claim 1, wherein the determining comprises obtaining a quantitative measure associated with the true contour of the movement at a plane containing the point-of-interest within the target volume.

6. The method of claim 1, wherein the measuring is facilitated by at least one of a digital ruler/scale, contouring tool, and a reference graphic overlay provided with the combined view, the contouring tool providing functionality to indicate an extent of a motion in the combined view.

7. The method of claim 1, further comprising:
changing an aspect of the computed tomography scanner to target volume geometric configuration by changing a position of a couch of the computed tomography scanner;
obtaining a second reference projection image of a target volume at the isocenter of the computed tomography scanner, in an instance the reference image is a fully divergent projection;
obtaining a second plurality of two-dimensional fluoroscopic images by the computed tomography scanner of at least a second portion of the target volume at the isocenter of the computed tomography scanner;
displaying the second reference projection image and the second plurality of two-dimensional fluoroscopic images in a second combined view, the second reference projection image forming a background of the second combined view and the second plurality of two-dimensional fluoroscopic images forming a foreground of the second combined view;
measuring a two-dimensional contour of a projection of a movement of the target volume in the second combined view; and
determining a true contour of the movement at a plane containing a point-of-interest within the target volume based on the two-dimensional contour of the projection of the movement in the second combined view.

8. A non-transitory medium storing processor-executable program code, the medium comprising:
program code to obtain a reference projection image of a target volume at an isocenter of a computed tomography scanner;
program code to obtain a plurality of two-dimensional fluoroscopic images by the computed tomography scanner of at least a portion of the target volume at the isocenter of the computed tomography scanner;
program code to display the reference projection image and the plurality of two-dimensional fluoroscopic images in a combined view, the reference projection image forming a background of the combined view and the plurality of two-dimensional fluoroscopic images forming a foreground of the combined view;
program code to measure a two-dimensional contour of a projection of a movement of the target volume in the combined view; and
program code to determine a true contour of the movement at a plane containing a point-of-interest within the target volume based on the two-dimensional contour of the projection of the movement.

9. The medium according to claim 8, further comprising program code to receive an indication of the point-of-interest at the isocenter within the target volume within a three-dimensional computed tomography volume proximal to the moving target volume, the coordinates of the point-of-interest within the target volume being known.

10. The medium of claim 8, wherein the reference projection image comprises at least one of a two-dimensional computed tomography projection radiograph and a digitally reconstructed radiograph.

11. The medium of claim 8, wherein the reference projection image and the plurality of two-dimensional fluoroscopic images are obtained at a same computed tomography scanner to target volume geometric configuration.

12. The medium of claim 8, wherein the determining of the true contour comprises obtaining a quantitative measure associated with the true contour of the movement in a plane containing the point-of-interest within the target volume.

13. The medium of claim 8, further comprising program code to provide at least one of a contouring tool and a reference graphic overlay with the combined view to facilitate the measuring, the contouring tool providing functionality to indicate an extent of a motion in the combined view.

14. The medium of claim 8, further comprising:
program code to obtain, in an instance the reference image is a fully divergent projection, a second reference projection image of a target volume at the isocenter of the computed tomography scanner for a new computed tomography scanner to target volume geometric configuration due to a change in position of a couch of the computed tomography scanner;
program code to obtain a second plurality of two-dimensional fluoroscopic images by the computed tomography scanner of at least a second portion of the target volume at the isocenter of the computed tomography scanner for the new computed tomography scanner to target volume geometric configuration;
program code to display the second reference projection image and the second plurality of two-dimensional fluoroscopic images in a second combined view, the second reference projection image forming a background of the second combined view and the second plurality of two-dimensional fluoroscopic images forming a foreground of the second combined view, in an instance the reference image is a fully divergent projection;
program code to measure a two-dimensional contour of a projection of a movement of the target volume in the second combined view; and
program code to determine a true contour of the movement in a plane containing a point-of-interest within the target volume based on the two-dimensional contour of the projection of the movement.

15. A system comprising:
a computed tomography scanner to obtain a reference projection image of a target volume at an isocenter of a computed tomography scanner and to obtain a plurality of two-dimensional fluoroscopic images of at least a portion of the target volume at the isocenter of the computed tomography scanner; and
a computing device to display the reference projection image and the plurality of two-dimensional fluoroscopic images in a combined view, the reference projection image forming a background of the combined view and the plurality of two-dimensional fluoroscopic images forming a foreground of the combined view; to measure a two-dimensional contour of a projection of a movement of the target volume in the combined view; and to determine a true contour of the movement in a plane containing a point-of-interest within the target volume based on the two-dimensional contour of the projection of the movement.

16. The system of claim 15, wherein the computing device further receives an indication of the point-of-interest within the target volume within a three-dimensional computed tomography volume proximal to the moving target volume, the coordinates of the point-of-interest within the target volume being known.

17. The system of claim 15, wherein the reference projection image comprises at least one of a two-dimensional computed tomography projection radiograph and a digitally reconstructed radiograph.

18. The system of claim 15, wherein the reference projection image and the plurality of two-dimensional fluoroscopic images are obtained at a same computed tomography scanner to target volume geometric configuration.

19. The system of claim 15, wherein the determining of the true contour comprises obtaining a quantitative measure associated with the true contour of the movement in a plane containing the point-of-interest within the target volume.

20. The system of claim 15, wherein the computing device further provides at least one of a contouring tool and a reference graphic overlay with the combined view to facilitate the measuring, the contouring tool providing functionality to indicate an extent of a motion in the combined view.

21. The system of claim 15, further comprising:
the computed tomography device obtaining, in an instance the reference image is a fully divergent projection, a second reference projection image of a target volume at the isocenter of the computed tomography scanner and obtaining a second plurality of two-dimensional fluoroscopic images of at least a second portion of the target volume at the isocenter of the computed tomography scanner, for a new computed tomography to target volume geometric configuration due to a change in position of a couch of the computed tomography scanner; and
the computing device displaying the second reference projection image and the second plurality of two-dimensional fluoroscopic images in a second combined view, the second reference projection image forming a background of the second combined view and the second plurality of two-dimensional fluoroscopic images forming a foreground of the second combined view; measuring a two-dimensional contour of a projection of a movement of the target volume in the second combined view; and determining a true contour of the movement within a point-of-interest of the target volume based on the two-dimensional contour of the projection of the movement in the combined second view.
